(19) United States
(12) Patent Application Publication
(10) Pub. No.: US 2008/0091140 A1
(43) Pub. Date: Apr. 17, 2008

(54) Cardiac Reperfusion Methods and Devices

(76) Inventor: Jaap Nico Hamburger, Vancouver (CA)

Correspondence Address: MOORE & VAN ALLEN PLLC, P.O. BOX 13706, Research Triangle Park, NC, 27709, US

(21) Appl. No.: 11/550,174

(22) Filed: Oct. 17, 2006

(30) Foreign Application Priority Data
Oct. 17, 2006 (CA) 2,564,263

(51) Int. Cl. A61M 25/00

(52) U.S. Cl. 604/93.01

(57) ABSTRACT

There are disclosed methods for preventing reperfusion injury, as well as apparatus and materials for the carrying out of the methods, and uses of the apparatus. In some embodiments the apparatus may take the form of a member which may be a guide wire, which may comprise delivery holes and markers and may be comprised in a catheter assembly. The methods and apparatus may be used to prevent or mitigate the reperfusion injury associated with the clearing of vascular occlusions

### BACKGROUND TO THE INVENTION

When reperfusing tissue, such as when a vascular occlusion is cleared, there is a risk of reperfusion injury distal to the occlusion. Animal studies have suggested the possible efficacy of chloramphenicol in the prevention of such injuries but systemic use in humans has been generally seen as ineffective.

A number of catheter and guidewire designs are already known in the art: U.S. Pat. No. 7,033,325 Sullivan discloses a guidewire comprising multiple radio opaque marker sections; U.S. Pat. No. 7,074,231 Jang, discloses a Convertible catheter system comprising a guidewire; and U.S. Pat. No. 4,946,466 Pinchuki & Martin discloses a transluminal angioplasty apparatus comprising a hollow guidewire. U.S. Pat. No. 4,994,033 discloses an intravascular catheter, and its use to apply medicament to a stenotic lesion in a blood vessel. U.S. Pat. No. 4,946,466 discloses a hollow guide wire with a diameter of about 0.014 inches, a balloon section and a hole to inflate the balloon and the use of fluoroscopy to view the progress of the metallic guide wire during insertion.

### SUMMARY OF THE INVENTION

In an embodiment there is disclosed a hollow member for the intravascular delivery of an agent, the member may have an end and a delivery hole proximate said end. In some embodiments there may be a marker positioned relative to the delivery hole.

In alternative embodiments, the member may be comprised in a catheter assembly, may be a guide wire, may be comprised in an apparatus for performing an angioplasty or the member may be filled with the agent.

In alternative embodiments, the member may be selected from the group consisting of: i) a cytochrome inhibitor; ii) a platelet activation factor inhibitor; iii) a Caspase inhibitor; and iv) a promoter of NO production.

In alternative embodiments, the intravascular delivery may be in a cardiac blood vessel; or may be in a blood vessel in the brain; or the member may be for use in a human.

In alternative embodiments, there is disclosed a method for reperfusing tissue, the method may comprise locally delivering an agent to the tissue prior to reperfusing the tissue.

In alternative embodiments, the method may further comprise controlling the amount of the agent delivered to the tissue; or exposing the tissue to the agent for a predetermined time period prior to the reperfusing; or the agent may be delivered through an intravascular member; or the member may be comprised in a catheter assembly.

In alternative embodiments, the member may include a marker and the method may further comprise using the marker to determine the location of delivering the agent.

In alternative embodiments, the method may further comprise: i) delivering the agent at a location relative to a vascular occlusion; and then ii) clearing the vascular occlusion.

In alternative embodiments, the occlusion may have first and second occlusion ends and the intravascular member may be inserted through the occlusion from the first end and used to dispense the agent at the second end.

In alternative embodiments, the member may be a guide wire; the guide wire may be comprised in a catheter assembly; or the guide wire may comprise a plurality of delivery holes; or may comprise a second marker; or may have an end, a delivery hole located relative to the end, a marker located relative to the delivery hole, and methods may further comprise using the marker to position the member relative to the tissue.

In alternative embodiments, the agent may be a chemical; and the chemical may be selected from the group consisting of: i) a cytochrome inhibitor; ii) a platelet activation factor inhibitor; iii) a Caspase inhibitor; and iv) a promoter of NO production; and v) a 2b3A receptor antagonist

In alternative embodiments, there is disclosed a kit for reperfusing tissue, the kit may comprise: i) a hollow member suitable for intravascular delivery of a protective agent; and ii) instructions to use the member to locally deliver an agent prior to reperfusing the tissue; the kit may further comprise instructions to fill the member with the agent prior the reperfusing; and the kit may further comprise instructions to allow the agent to contact the tissue for a predetermined time prior to the reperfusion.

In alternative embodiments, there is disclosed use of a hollow member for the local delivery of an agent for the prevention of reperfusion injury; and the member may be adapted to be insertable through a vascular lumen; and may have a delivery hole and a marker positioned at a defined location relative to the delivery hole.

In alternative embodiments, the use may comprise filling the member with the agent; or may comprise dispensing a known quantity of the agent through the member; or may comprise allowing a determined time to pass between the delivery of the agent and reperfusing the tissue.

In alternative embodiments, the member may comprise a plurality of the markers and a plurality of the delivery holes.

In alternative embodiments, the reperfusion may be reperfusion of a cardiac blood vessel; or of a blood vessel in the brain.

In alternative embodiments, the agent may be selected from the group consisting of: i) a cytochrome inhibitor; ii) a platelet activation factor inhibitor; iii) a Caspase inhibitor; iv) a promoter of NO production; and v) a 2b3A receptor antagonist.

In alternative embodiments, there is disclosed use of a catheter assembly having a hollow guide wire for the reperfusion of tissue associated with the clearing of a vascular occlusion, use may comprise locally delivering a chemical agent through the guide wire prior to the clearing of the vascular obstruction.

In alternative embodiments, there is disclosed use of a hollow member having an end, a delivery hole positioned proximate the end, and a marker positioned relative to the delivery hole to manufacture an apparatus for the local intravascular delivery of a agent in the removal of a vascular occlusion.

In alternative embodiments, there is disclosed use of a locally administered agent for prevention and treatment of reperfusion injury.

In alternative embodiments, there is disclosed use of a locally delivered agent for preventing and mitigating, and treating reperfusion injury in a human cardiac blood vessel, the use may comprise sequentially: i) delivering a desired quantity of the agent at a desired intravascular location through a hollow intravascular member; and ii) waiting a desired time interval after the local delivery; and then iii) carrying out the reperfusion.

In alternative embodiments, there is disclosed a method for clearing a vascular occlusion in a cardiac blood vessel, the method may comprise sequentially: i) exposing tissue affected by the occlusion to a desired quantity of an agent for a desired time; and then ii) reperfusing the tissue.

In alternative embodiments, there is disclosed a method for clearing a vascular occlusion using a catheter assembly with a hollow guide wire, the method may comprise sequentially: i) passing the guide wire through the occlusion; ii) delivering a desired quantity of an agent through the guide wire; iii) waiting for a desired time; and iv) clearing the vascular occlusion.

In alternative embodiments, there is disclosed a method for intravascularly delivering a agent for use in reperfusion, the method may comprise: i) filling a hollow member with the agent; ii) feeding the member through a desired blood vessel; iii) expelling a desired quantity of the agent from the member at a desired location.

In a further embodiment, there is disclosed an apparatus for the intravascular delivery of an agent the apparatus comprising a fluid pressure source and a member, the member having an end and a delivery hole positioned relative to the end and the fluid pressure source being operatively connected to the member.

In further embodiments, the apparatus may be a catheter assembly.

In a further embodiment, there is disclosed a method for preparing tissues prior to reperfusion the method comprising delivering an agent to the tissue prior to reperfusing the tissue.

In further embodiments, the method may further comprise controlling the amount of the agent delivered to the tissue; or may further comprise exposing the tissue to the agent for a predetermined time period prior to the reperfusing; or the agent may be delivered through an intravascular member; or the member may be comprised in a catheter assembly.

In further embodiments, the member may include a marker and a delivery hole and the method may further comprise using the marker to determine the location of the delivery hole.

In further embodiments, the method may further comprise: i) delivering the agent at a location relative to a vascular occlusion; and ii) clearing the vascular occlusion.

In further embodiments, the method may include clearing a vascular occlusion and the occlusion has first and second occlusion ends and the intravascular member is inserted through the occlusion from the first end and used to dispense the agent at the second end.

In further embodiments, the members disclosed may be used to prevent reperfusion injury; and the uses may occur as part of an angioplasty procedure; the member may be filled with the agent; or may be a guide wire; or may have an associated marker.

In further embodiments, the guide wire may have an end, and a delivery hole located relative to the end, and the method may further comprise positioning the member relative to the tissue to be reperfused. Blood vessels may be blood vessels in the brain, or may be coronary blood vessels and tissue may be human tissue. Features and advantages of the embodiments will become more apparent in light of the following detailed description of some embodiments thereof, as illustrated in the accompanying figures. As will be realized, the subject matter hereof is capable of modifications in various respects, all without departing from the scope of the disclosure and claims. Accordingly, the drawings and the description are to be regarded as illustrative in nature, and not as restrictive.

### BRIEF DESCRIPTION OF THE DRAWINGS

FIG. 1 is a perspective view of a first embodiment of a member.

FIG. 2 is a side view of the embodiment of FIG. 1.

FIG. 3 is a cross sectional side view of the embodiment of FIG. 1.

FIG. 4 is a top view of the embodiment of FIG. 1.

FIG. 5 is an end view of the embodiment of FIG. 1.

FIG. 6 shows a second embodiment of a member.

FIG. 7 shows a third embodiment of a member.

FIG. 8 is a diagram showing the possible positioning of a guidewire according to an embodiment.

FIG. 9 is a diagram of a catheter assembly according to an embodiment.

FIG. 10 shows the arrangement of syringes of an embodiment.

FIG. 11 shows a sketch of the spread of Evans Blue in a heart following perfusion through a member of an embodiment.

### DETAILED DESCRIPTION OF THE EMBODIMENTS

### Definitions

In this application the following terms have the following meanings:

“Agent” (which may also be referred to as “protective agent”, or “preventive agent”) means any species which is able to prevent, mitigate, reduce, or control reperfusion injury. Such active species may include chemical agents, biologics, and nanoparticles and may be pharmaceuticals or other bioactive chemicals, and may include macromolecules, hormones, signalling molecules, organic and inorganic compounds, micro organisms and other agents. By way of illustration and not of limitation, possible agents for use in the embodiments disclosed herein may include (a) inhibitors of superoxide production which may be superoxide dismutases, catalase, iron chelating agents such as deferoxamine and may be cytochrome inhibitors and may be cytochrome p450 inhibitors, and may be chloramphenicol; (b) Platelet Activation Factor (PAF) inhibitors such as TCV309™; (c) Caspase inhibitors; (d) promoters of NO production such as nitroprusside and adenosine; and (e) 2b3A receptor antagonists. Suitable agents and suitable dosages for such agents will be readily apparent to those skilled in the art. In alternative embodiments the agent may be dissolved or may be in suspension or may be provided in an aqueous or other medium and may be provided at any suitable concentration in any suitable medium or carrier or form and may be provided in association with or may include any suitable carriers or recipients. It will further be understood that particular agents may be used in combinations with other agents or with other components.

“Agent fluid pressure source” means any apparatus, device or combination of elements suitable to provide a source of pressure for the delivery of agent through a member. Possible examples of suitable fluid pressure sources include syringes, pumps, elevated reservoirs, or any other types of dispenser. A range of alternatives will be readily understood and adapted by those skilled in the art who will readily be able to understand and make suitable choices there between and adjustments thereto.

“Angioplasty” means a surgical operation to repair a damaged blood vessel, or to unblock a blood vessel, and may also be known by other names including “coronary artery balloon dilation”, “balloon angioplasty” and “percutaneous coronary intervention” (PCI). The subject blood vessel may be an artery and in particular embodiments it may be a coronary artery or an artery in the brain. In particular embodiments an angioplasty may be carried out using a catheter apparatus, and the catheter apparatus may comprise a member that may be a guidewire.

“Catheter apparatus” has its normal meaning and includes an assembly comprising one or more hollow, flexible tubes that can be inserted into a body cavity, duct, or vessel to allow the passage of fluids or distend a passageway or carry out a range of procedures as will be readily understood by those skilled in the art. A wide range of catheter designs will be well known to those skilled in the art and those skilled in the art will be readily able to select appropriate designs and to adapt both procedures and designs for the carrying out of various embodiments.

“Delivery hole” means any hole, orifice, port, slit, crack, aperture, opening, space, gap, fissure, pore or micropore suitable for the delivery there through of an agent for use in an embodiment, it being understood that such holes may be of any suitable size and may be provided in a metal, or in a plastic or other suitable material, and may be created by any suitable methods including but not limited to laser or mechanical working of a substrate material or may be preformed in a material. It will be understood that in different embodiments such holes may be provided singly or in groups and may comprise any number of holes. It will be understood that in particular embodiments pluralities of holes may be organised in any regular or random fashion and may form a mesh or filter and may be positioned at a range of locations along a member. The choices between alternative designs will readily be made by those skilled in the art.

“Guidewire” has its usual meaning and includes for example a wire used in the treatment of a vascular occlusion, optionally as a part of a catheter assembly.

“Marker” means a defined region, tag, indicator, material or other marker suitable to permit an operator to localise a defined portion of a member. In particular embodiments the marker may be localised using fluoroscopy, or computer tomography in which cases the marker may generally be a radio opaque region having suitable properties. Suitable radio opaque materials may include by way of example gold, platinum, lead and tungsten but a range of alternative materials, shapes, constructions, and detection methods will be readily apparent to those skilled in the art. In alternative embodiments it may be possible to locate the markers using alternative techniques such as MRI in which case the markers may be magnet opaque or may comprise materials suitable to such alternative techniques, which materials may include copper. In particular embodiments the member may comprise multiple markers which have different shapes, opacity, or properties so that their relative positions may be determined by an operator.

“Member” means a member suitable for conducting an agent, and may be generally, or in part, tubular, and may include a conduit channel, hollow portion, reservoir, or other structure suitable for conducting a fluid. In some embodiments the member may be suitable for feeding along the lumen of a blood vessel, and may be coated, in whole or in part, with suitable materials to ensure its compatibility with the agent to be carried and to facilitate its movement through a blood vessel lumen. Typically the diameter of a member may be between 0.01 and 0.02 inches but in particular embodiments the diameter may range from less than 0.010 inches, 0.010-0.012 inches, 0.012-0.014 inches, 0.014-0.016 inches, 0.016-0.018 inches, 0.018-0.02 inches. In particular embodiments these diameters and diameters greater than 0.02 inches may all be possible and the choice of an appropriate dimension will be readily apparent to those skilled in the art. A member may be made from a range of materials and in a range of constructions all of which will be readily apparent to those skilled in the art. In particular embodiments the member may comprise flexible metals and in certain specific embodiments may comprise nitinol or stainless steel. In certain embodiments the member may be adapted to facilitate steerability, movement and flexibility whilst maintaining the integrity of the member and any conduit therein. In some embodiments the member may be a guide wire. In particular embodiments the member may be coated or treated with lubricious substances such as teflon, hydrogels or hydrophilic coatings. In some embodiments the member may have associated structures such as holes, scrapers, or a range of suitable implements to permit the member to be advanced through a vascular occlusion.

“Reperfusion” means the restoration of blood flow to an ischemic organs, tissues, or cells.

“Reperfusion injury” means injury resulting from the reperfusion of tissue and may result from sudden exposure of ischemic organs, tissues or cells to oxygenated blood; it may include oxidative stress, apoptosis, inflammation, and ischemic injuries.

Those skilled in the art will readily understand and implement a variety of methods whereby suitable agents may be applied through the members disclosed, and will understand that a wide range of catheter designs are possible and will readily choose suitable designs.

### Description of Specific Embodiments

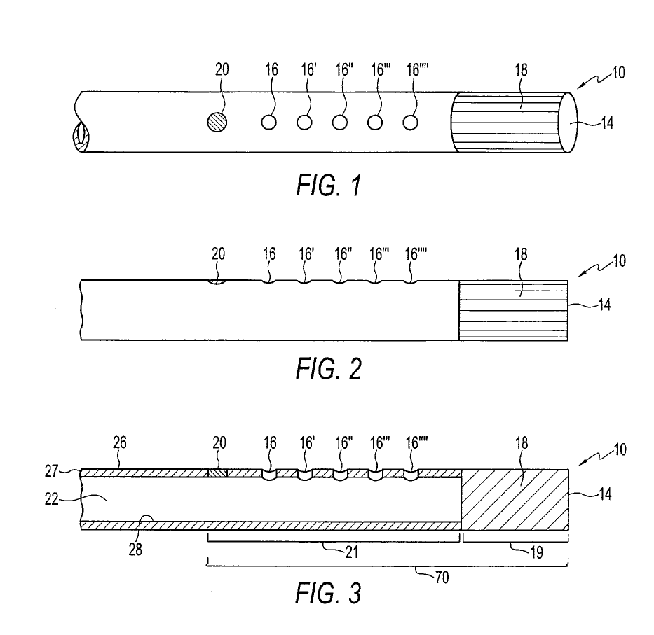

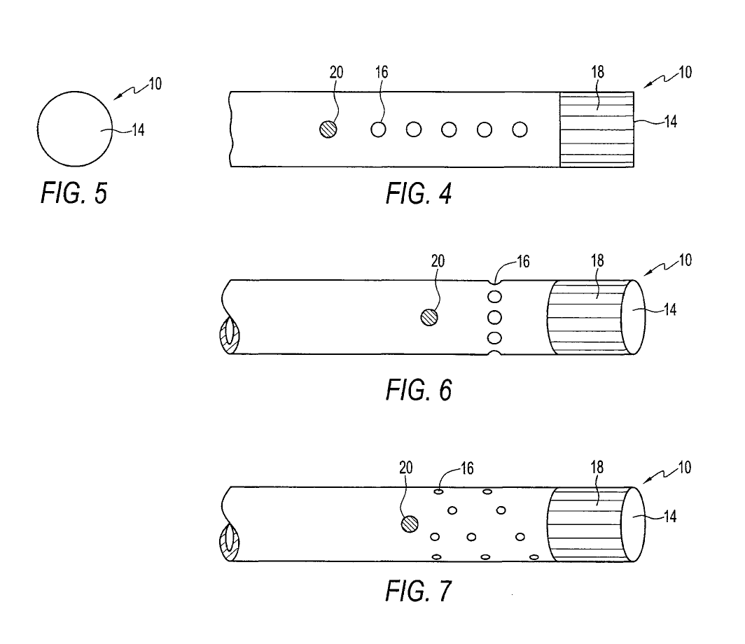

In a first embodiment described with reference to FIGS. 1, 2, 3, 4 and 5, there is disclosed a hollow member generally designated 10, for the intravascular delivery of an agent. The member has an end 14, and at least one delivery hole 16. The at least one delivery hole 16 is positioned relative to the end 14 of the member, and a marker 18 is positioned relative to the delivery hole and proximate end 14. In the illustrations marker 18 is a radio opaque material making up material over length 19 the end 14 of the member and may comprise platinum. It will be appreciated that a range of alternative configurations and materials are possible. In the embodiment illustrated the marker 18 has a length 19 of from about 20 mm to about 30 mm, but in alternative embodiments the length 19 may be adjusted in ways that will be readily apparent to those skilled in the art, so as to maintain a suitable balance of stiffness and flexibility for this portion of the member. Particular elements of the apparatuses disclosed herein may be within a region generally designated 70 at the end of the member 10.

In this embodiment a second marker 20 is presented on the opposite side of the delivery holes 16 from first marker 14, it will be appreciated that in alternative embodiments the first maker, the second marker, or both markers may be omitted, or that additional markers or alternative marker placements may be adopted or that the markers may have the same or different shapes and properties. The location of one or both markers 18, 20 may be known relative to the delivery holes 16. Although the embodiments illustrated have markers 18 and 20 positioned on either side of the delivery holes 16, alternative arrangements, or the omission of one of the markers, may be possible in particular embodiments.

As will be seen, although closed at end 14 by marker 18, member 10 comprises a lumen 22 defined by a wall 27 and communicating with delivery holes 16.

The member 10 will be compatible with the agent to be dispensed, and the interior surface 26 of the member may optionally be coated with suitable materials for this purpose. In practice, prior to use, the member may be flushed with heparin, or with heparin saline or other anticoagulants. The exterior surface 28 of the member may optionally be coated with suitable lubricious substances to facilitate movement of the member 10 through the lumen of a blood vessel. Although the embodiment illustrated in FIGS. 1-5 comprises five delivery holes 16, and two markers, it will be appreciated that any number of holes, and any number of markers may be used to suit particular requirements. In particular alternative embodiments illustrated in FIGS. 6 and 7 the delivery holes 16 may be arranged in alternative patterns around the member 10 and in a range of sizes as set out herein. The only requirement may be that the agent is releasable through the delivery holes in a controlled manner. It will be understood by those skilled in the art that the number, size and distribution of the delivery holes should be adjusted to allow a suitable flow rate of agent out of the member, so as to permit rapid delivery of agent without damaging the interior of a blood vessel or cavity into which it is inserted.

Although in the first illustrated embodiment the length 19 of marker 18 may be between about 20 mm and about 30 mm, a wide range of alternative sizes may be possible, for example, lengths 19 of from about 5 mm to about 10 mm, about 10 mm to about 15 mm, about 15 mm to about 20 mm, about 20 mm to about 25 mm, about 25 mm to about 30 mm, about 30 mm to about 40 mm or greater than about 40 mm may all be possible and suitable under particular circumstances. Likewise the size of second marker 20 may be changed and a range of suitable sizes may be possible. In the first embodiment marker 20 and marker 18 may be radio opaque and may be or may comprise gold or platinum but other metallic or non-metallic substances may be suitable and will be readily chosen by those skilled in the art to suit particular purposes. In the first embodiment distance 21 between markers 18 and 20 may be about 10 mm, but again a range of sizes may be possible, depending on operational requirements, and the number and disposition of delivery holes 16.

The member itself may be of conventional length, as determined by those skilled in the art, and may be sufficient to extend from a desired point of introduction to the vascular system to the identified target location whilst still leaving sufficient length outside the body for necessary manipulations.

In certain alternative embodiments there is also disclosed an apparatus comprising a member and a fluid pressure source.

In alternative embodiments the member may be adapted for us in a mammal, which may be a human. It will be appreciated that in certain embodiments the markers may be omitted entirely and the physical or radiographic properties of the member as a whole may be used to localize the end of the member. In some uses those skilled in the art will be able to localize and use the member without recourse to radiography or fluoroscopy and may rely on physically locating the end of the member manually, by feeling its location in the subject blood vessel.

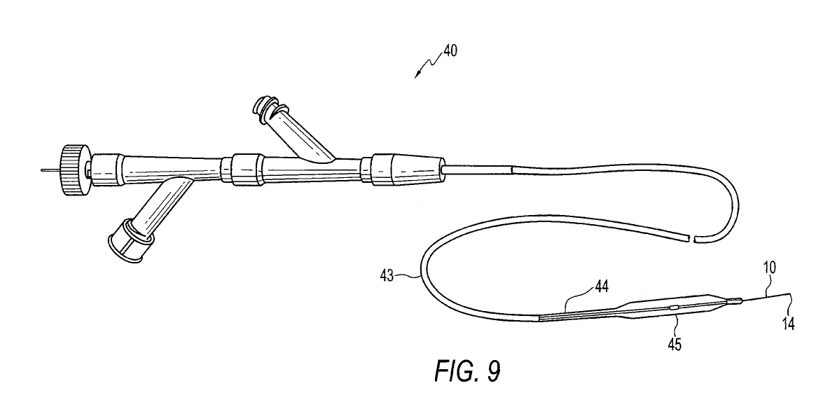

In a second embodiment there is disclosed the use of a catheter assembly having a hollow member 10, for the reperfusion of tissue associated with the clearing of a vascular occlusion. The vascular occlusion may be in a cardiac artery. An example of a catheter assembly is presented in FIG. 9 and generally designated 40. The member is designated 10 and has an end 14. Catheter sheath 43 surrounds a further catheter element 44 having associated balloon member 45. The use of the assembly may comprise a series of steps, the details of which will be readily adapted by those skilled in the art to suit particular patients, particular objectives, alternative catheter designs and other variables.

a) Before use, the member 10 (which may be a guide wire), may be first flushed and washed with heparin or heparin saline or other suitable anticoagulants, and then with agent.

Before use the member may be filled with agent.

b) Necessary preparation for the procedure may vary with the condition and location of the subject. A variety of routine procedures and precautions that may be implemented prior to, during and after the operation will be readily apparent to those skilled in the art who will be able to make appropriate choices there between. These include but are not limited to the following:

The subject may be asked to stop eating or drinking for a suitable time before the operation, and in certain embodiments such suitable time may be up to 12 hours or more than 12 hours; Routine tests may be carried out before commencing the procedure, these may include: chest X-rays, electrocardiograms and blood tests; Prior to the procedure it may also be desirable that the subject stop taking certain medications which may include those for diabetes; The subject's heart rate and rhythm may be monitored during the procedure using a variety of known techniques.

General anesthesia may or may not be used depending on the circumstances. It may be desirable to administer anticoagulants to reduce blood clotting and suitable medications to relax the blood vessels that are to be treated. Subjects may also be given calcium blockers, nitrates or other suitable medications to reduce any risk of vascular spasm.

c) Briefly: A needle may first be inserted, then a wire there through. A sheath may be guided into the blood vessel over this wire, the sheath may contain a one way hemostatic valve to prevent blood flow out of the blood vessel. Then the needle may be removed and a sheath advanced along the wire into the blood vessel and then the wire removed A guiding catheter 43 with a wire may then be advanced along the blood vessel up to the coronary artery. Then a third intravascular or coronary guide wire may be introduced and may be used to deliver an agent and to guide a coronary catheter to the site of treatment

An exemplary insertion point to access the coronary artery may be at the femoral artery but in some embodiments the brachial or radial arteries, or other blood vessels, may also be suitable. Where the femoral artery is used, the insertion site may be in the groin. Before the procedure begins, the area for inserting the catheter may be prepared with antiseptic solution and a local anesthetic may be administered. In some embodiments a suitable needle is inserted into a chosen blood vessel and a guide wire introduced there through. The needle may then be removed and a catheter sheath may slipped over the wire and into the artery and then (where this is the target) a guiding catheter may be manipulated into a coronary artery. The catheter which is advanced to the coronary artery may be introduced through a vascular access sheath and may be a separate catheter from that initially introduced to the blood system. Alternatively the catheter sheath may be the first element introduced over a wire, following puncture with a needle. In alternative embodiments, insertion of the sheath may precede insertion of the guide catheter which may precede insertion of the guide wire, insertion of the guide wire may precede the sheath, or alternative sequences may be adopted. It will be understood that different designs of guide wires may be used at different stages in the procedure: for introduction of a vascular access sheath; for advancing a guiding catheter towards the target artery which may be a coronary artery; and finally, intracoronary for guidance of coronary intervention catheters (balloons, stents, etc). In particular embodiments disclosed the member will be of the latter variety, an intracoronary guide wire for guidance of an intervention catheter.

d) The member 10 may be advanced to a desired location, in some embodiments this may be the entrance to the coronary artery. The catheter operator may use x-ray images to follow the location of the guide catheter 43 and or member 42, until it reaches the target which may be a blocked coronary artery. The catheter operator may inject a small amount of contrast agent, which may contain iodine, or dye, through the catheter to help in following the location of the catheter.

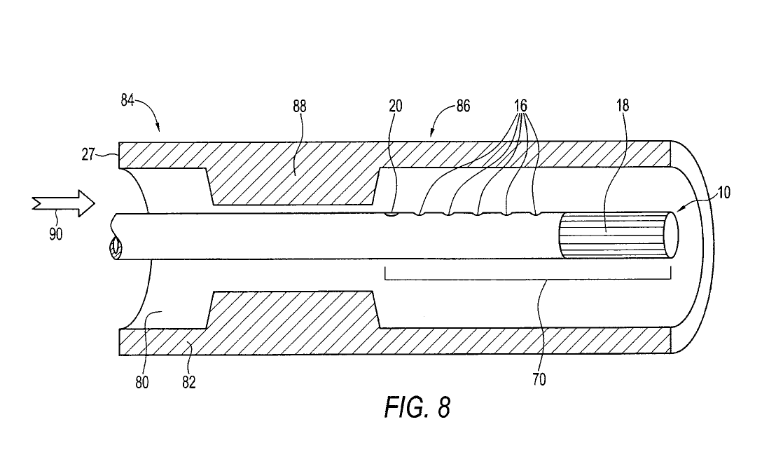

e) The member 10 may be advanced through the catheter to the location of a target vascular occlusion and then advanced through the occlusion to a position where it may be used to dispense the desired agent downstream of the occlusion. FIG. 8 shows the placement of the end section 70 of a member 10 when positioned to deliver an agent in one embodiment. It will be observed that in this embodiment the member is disposed in the lumen 80 of a blood vessel 82, and extends from an upstream side 84 to a downstream side 86 of an occlusion 88. Agent can thus be delivered from delivery holes 16 downstream of the occlusion. It can be seen that the end 70 of the member 10 can be positioned relative to the occlusion 88 by reference to one or both of the markers 18, 20. Such positioning may occur prior to, during or after the end of the member is advanced through the occlusion. The direction of blood flow is shown by arrow 90. The location of an end portion 70 of the member may be determined by x-ray techniques, fluoroscopy or any other suitable means. In one embodiment the agent may be chloramphenicol and about 10 mg may be administered in about 0.5-10 ml volume. A wide range of suitable alternative agents (as defined herein) may be possible and will be readily selected, and dosages readily determined, by those skilled in the art.

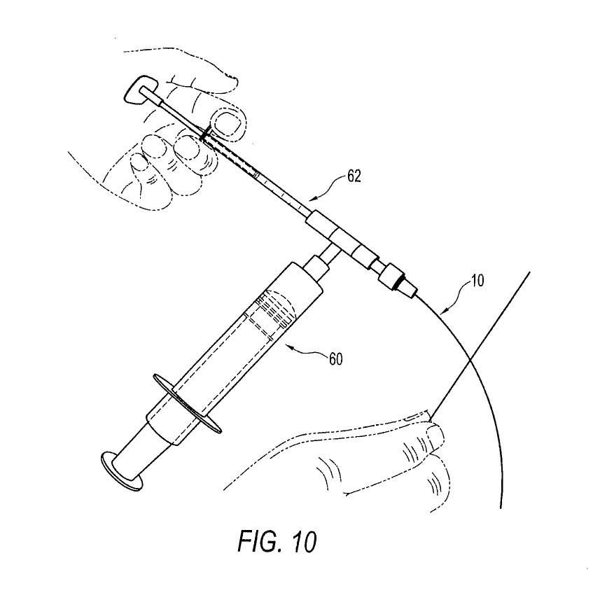

FIG. 10 illustrates a suitable arrangement of syringes for the controlled delivery of an agent through a member. Two syringes 60 and 62 are interconnected as shown by a three way stop-cock which is adjusted as necessary to allow the desired flow of agent. Agent is transferred from a reservoir syringe 60 into a dispensing syringe 62 by compression of reservoir syringe 60. As desired dispensing syringe 62 may be compressed to urge agent into the lumen of member 10 in a desired quantity and rate. It will be appreciated that a range of suitable one-way valves or other control devices may be provided between syringe 60 and 62 and between syringe 62 and member 10 as desired.

f) After delivering the agent the operator of the catheter may wait for a predetermined time before clearing the occlusion, in some embodiments this may be about one minute but as disclosed herein alternative time periods are possible. After a suitable waiting period a balloon member 65 or other device may be used to clear the occlusion. In an embodiment a suitable balloon may be inflated for a period that may be up to a minute at the site of the blockage, in alternative embodiments this inflation step may be repeated and the procedure may be repeated at the sites of a plurality of blockages or the duration of the inflation step may be shortened or extended in ways readily apparent to those skilled in the art.

g) In some embodiments, once the occlusion has been cleared, a stent or stents may be placed in the artery using a range of known procedures. Once a stent or stents are in place, the balloon catheter may be removed and angiograms may be taken to see how well blood flows through the cleared artery. In alternative embodiments the blood vessel may be widened before, during, or after the stent has been opened up.

h) The various parts of the apparatus may be withdrawn from the patient according to standard procedures. In particular alternative embodiments the entire procedure usually may take about 30 minutes to several hours. The sheath may be left in place for several hours after the procedure. The entry area may be kept immobile until the sheath has been out for an extended period which may be up to or more than about three hours. A range of standard procedures may be adopted to reduce or prevent bleeding and infection. It may be desirable to monitor the patient's heart and vital signs for 12 to 24 hours after the procedure, and it may be desirable to continue treating with relaxants and anticoagulants. The patient may remain hospitalized for one or more days. After the procedure it may be desirable for the patient to drink plenty of fluids to help rid their body of the contrast dye and to avoid strenuous exercise and lifting heavy objects for several days afterward.

It will be appreciated that in some embodiments the member 10 may be a guide wire, or may take some other form and that in some embodiments the apparatus may include different combinations of sheath elements, guide wire elements, guide catheter elements, balloon catheter elements etc, and that in particular elements one or more of such elements may be omitted. It will also be appreciated that in alternative embodiments the sequence of placing and advancing the different elements may be changed, or as indicated some elements or their use may be omitted altogether. The choice between different catheter designs and different usage procedures will be readily made by those skilled in the art. As described above, in certain embodiments the member may not have associated markers.

### Alternative Embodiments

A range of alternatives to the use of interconnected syringes will be readily identified and implemented by those skilled in the art and may include computer controlled dispensing apparatuses, premeasured dispensers, or any other mechanism suitable to expel a controlled quantity of agent from the guide wire, at a suitable rate.

In particular alternative embodiments the desired dosage of agent may vary with the subject, the circumstances and the particular agent selected. When the agent is chloramphenicol a dosage of between about 5 mg and about 15 mg may be suitable, if the agent is adenosine a dosage of between about 30 ug and 50 ug may be suitable. Generally the agent may be delivered in a volume of about 0.5 ml to 1.0 ml, but larger or smaller volumes may also be used and suitable parameters will be readily determined by a suitably skilled user. In alternative embodiments the method may further comprise controlling the amount of agent delivered to the tissue in relation to parameters that will be readily apparent to those skilled in the art. It will be understood that the desired dosage may change with the nature of the tissue to be reperfused, the time for which the tissue has been denied oxygen, the agent used, the temperature of the tissue, the composition of the reperfusion fluid and other parameters, all of which will be readily understood and evaluated by those skilled in the art. Suitable dosages for any chosen agents will be readily determined by those skilled in the art to suit particular circumstances.

In particular embodiments the tissue to be reperfused may be exposed to the agent for any suitable period of time prior to the reperfusion step. In some embodiments the tissue may be exposed to the agent for about 1 minute prior to reperfusion, but in particular embodiments this time period may be up to 1 second, up to 2 seconds, up to 5 seconds, up to 10 seconds, up to 20 seconds, up to 30 seconds, up to 40 seconds, up to 50 seconds, up to 60 seconds, up to 80 seconds, up to 100 seconds, up to 120 seconds, up to 140 seconds, up to 160 seconds, up to 180 seconds or more than 180 seconds. In certain embodiments t the agent may be delivered to the tissue only a minimal amount of time in advance of the reperfusion step.

In particular embodiments the methods and apparatuses and uses disclosed herein may be directed at the treatment, or the prevention or the treatment and prevention of reperfusion injuries.

The method and apparatus of particular embodiments may each be directed at the treatment of an occlusion or the reperfusion of tissue, or both, in a cardiac blood vessel, which may be a cardiac artery. Alternatively they may be directed at other blood vessels including blood vessels in the brain. In some embodiments an occlusion may be approached from an upstream direction but it will be appreciated that in alternative circumstances, such as during an operation, the agent may be directly introduced to the region downstream of an occlusion, may be approached from the downstream side of the occlusion through a blood vessel lumen, and may be approached by injection directly through the blood vessel wall. In one alternative embodiment the agent may be introduced directly into the coronary sinus. In further alternative embodiments the method may comprise simply locally delivering an agent to the tissue prior to reperfusing the tissue. In further alternative embodiments apparatuses and methods disclosed may be used in the reperfusion of tissue in any suitable blood vessels, including blood vessels in the brain and alternative cardiac blood vessels. It is also envisaged that in some embodiments the agent may be deposited upstream of the occlusion, and a temporary hole introduced in the occlusion allowing the upstream blood pressure to force the agent through the hole to the other side of the occlusion.

The various embodiments may be provided in the form of kits for reperfusing tissue. These may comprise a hollow member for intravascular delivery of an agent and exemplified in FIGS. 1-7, and in the specific embodiments disclosed herein, as well as instructions to use the member to locally deliver an agent prior to reperfusing tissue. In alternative embodiments a kit may further comprise instructions to fill the member with agent prior reperfusing tissue. In further alternative embodiments a kit may further comprise instructions to allow the agent to contact the tissue for a predetermined time prior to the reperfusion.

In further embodiments a hollow member may be used for the local delivery of an agent for the prevention of reperfusion injury. In yet further alternative embodiments the member may be adapted to be insertable through a vascular lumen. Uses of the member may comprise filling the member with an agent, may comprise dispensing a known quantity of an agent through the member and may comprise allowing a determined time to pass between the dispensing of an agent and reperfusing the tissue In alternative embodiments the member may be comprised in a catheter assembly which may include one or more markers and the one or more markers may be used to determine the location of delivering the agent. In further alternative embodiments the method may further comprise delivering the agent at a location relative to a vascular occlusion and then clearing the vascular occlusion; the occlusion may have first and second occlusion ends and the intravascular member may be inserted through the occlusion from the first end and used to dispense the agent at the second end. In alternative embodiments the member may be a guide wire and the guide wire may be comprised catheter assembly which may be an over the wire catheter assembly or a rapid exchange catheter assembly or a mono-rail catheter assembly. In further refined embodiments the guide wire may comprise an end, a delivery hole located relative to the end, a marker located relative to the delivery hole, and the method may further comprise using the marker to position the member relative to the tissue. In alternative embodiments of the method the guide wire may comprise more than one delivery holes, or may comprise more than one marker, or may comprise more than one delivery hole and more than one marker. In particular embodiments the agent may be a chemical, and may be an inhibitor of superoxide production, may be a cytochrome inhibitor; may be a platelet activation factor inhibitor; may be a Caspase inhibitor; may be a 2b3A receptor antagonist and may be a promoter of NO production. In some embodiments the reperfused tissue may be associated with a cardiac blood vessel, or may be associated with a brain blood vessel, or may be any other suitable blood vessel. In particular embodiments the method may be carried out on a human subject in need thereof. In alternative embodiments locally administered chloramphenicol may be used for preventing reperfusion injury. The method of administration may include any of those methods set out in the alternative embodiments.

There is further disclosed the use of the member and variants of the various embodiments to prevent reperfusion injury. In alternative embodiments the use may be carried out as part of an angioplasty procedure and locally delivered agents may used for preventing reperfusion injury in a human cardiac blood vessel. The uses disclosed may comprise sequentially delivering a desired quantity of an agent at a desired intravascular location through a hollow intravascular member; and waiting a desired time interval after the local delivery; and then carrying out the reperfusion. In further embodiments there is further disclosed a method for clearing a vascular occlusion in a cardiac blood vessel. The method may comprise sequentially exposing tissue affected by the occlusion to a desired quantity of a suitable agent for a desired time; and then reperfusing the tissue. There is further disclosed a method for clearing a vascular occlusion using a catheter assembly with a hollow member. The method may comprise sequentially passing the member through the occlusion; delivering a desired quantity of a suitable agent through the member; waiting for a desired time; and clearing the vascular occlusion.

There is further disclosed a method for intravascularly delivering a agent for use in reperfusion. The method may comprise filling a hollow member with an agent; feeding the member through a desired blood vessel and expelling a desired quantity of the agent from the member at a desired location.

### EXPERIMENTAL EXAMPLES

The following examples are given by way of illustration and not limitation:

Background: Percutaneous coronary intervention (PCI) improves survival from myocardial infarction (MI). Ischemia-reperfusion injury (IRI) is an important factor influencing the outcome following MI. Systemic strategies for IRI reduction are suboptimal due to the coronary occlusion.

Methods: Female juvenile pigs (n=14) were used. All animals received 250 mg of aspirin and 100 U/Kg of heparin IV. Tying the mid Left Anterior Descending Artery (“LAD”) after distal TGT (Trans Guide wire Therapy) wire placement induced acute myocardial ischemia. The TGT system consisted of a 0.014 inch nitinol torquable guide wire and removable stopcock. The distal 30 mm tip was flexible and radio-opaque. Immediately proximal to the tip were delivery holes to facilitate TGT delivery. TGT was delivered after 30 minutes of ischemia. The distal coronary vascular bed was visualized by injection with radiographic contrast (n=2) and Evans blue (n=2). Drug effect was evaluated by comparison of TGT with 1 cc heparinized saline (n=5) or chloramphenicol 10 mg (1 cc) TGT with 40 mg/Kg IV (n=5). Suture removal allowed 2 hours of reperfusion prior to sacrifice. Hemodynamics expressed as the heart rate blood pressure product (RPP) was assessed continuously. Echocardiographic LV ejection fraction (LVEF) was measured at baseline, end of ischemia (pre-TGT) and pre-sacrifice. Device success was defined as successful TGT delivery.

Results: TGT was successfully performed in all animals. Luminal potency was maintained allowing instantaneous TGT injections. X-ray dye and Evans blue injection graphically demonstrated the TGT concept. The dye stained up to the LAD watershed demarcating the ischemic area at risk. TGT with chloramphenicol showed significantly better RPP (rate pressure product; pre-sacrifice; chloramphenicol vs. saline: 3720.0 (+/−510.2) vs. 2685.7 (+/−2227.6); p=0.02). Intravenous inotrope support was significantly less in the chloramphenicol group (chloramphenicol vs. saline (arbitrary units): mean 1.67 (+/−1.6) versus 7.0 (+/−5.1) vs. p=0.028). Left ventricular ejection fraction in TGT chloramphenicol-treated animals normalized during reperfusion, returning to near baseline (27.3%+/−3.8 to 43.3%+/−11.4, p<0.001). Saline-TGT animals did not recover from ischemia with just minimal improvement of EF (35.1%+/−12.2 to 38.9%+/−7.0, p=0.52).

Methods

Animal Model: All animals were maintained in accordance with the principles outlined in the Guidelines of the Canadian Council of Animal Care under the supervision of the animal care committee of The University of British Columbia.

Animal Preparation: Female juvenile domestic swine, weight 30-50 kg, were studied. Swine have sparse collateral circulation and do not form collaterals in response to acute ischemia. Anaesthesia was induced with an intramuscular injection of ketamine (20 mg/kg) followed by inhaled isoflurane (0.5-2.0%). After general anaesthesia the femoral artery was cannulated. A pulmonary artery cannula was positioned via the right internal jugular vein. All animals received 250 mg of IV aspirin and 100 U/Kg units of heparin intravenously to achieve an ACT above 250 s.

TGT (Trans Guide wire Therapy) Wire Description: The TGT system consisted of a 0.014 inch nitinol steerable guide wire and an attachable 3-way stop cock (FIG. 10). The wire tip was flexible and, if required, re-shapable during a procedure in order to meet specific anatomic circumstances. Furthermore, it had a torque device mounted on the proximal shaft. The distal 30 mm of the wire was relatively floppy and radio-opaque for visualization under fluoroscopy. A gold marker proximal to the radio-opaque part of the wire indicated the position of multiple exit ports. The body of the wire was treated with a hydrophilic coating.

Interventional Coronary and Surgical Procedures: A 6F Hockey stick guide catheter cannulated the left main coronary artery. The TGT wire, flushed with heparin, was positioned in the distal left anterior descending (LAD) artery. After a midline sternotomy acute regional myocardial ischemia was induced by occluding the mid portion of the LAD with a surgical suture. Fluoroscopy ensured the suture was above the infusion ports of the TGT wire with distal TIMI 0 flow. Lidocaine was given as a bolus and infusion at the onset of ischemia and for the remainder of the experiment. TGT injection was performed at the end of a 30 minute ischemic period after which the suture was removed to allow reperfusion. Restoration of TIMI 3 flow was documented by cine-angiography. Animals were sacrificed after 2 hours of reperfusion and the hearts were harvested for immunohistochemistry.

The 30 minute duration of ischemia was chosen because this is known to cause substantial damage (such as contraction band necrosis, coagulation necrosis and abundant infiltration of inflammatory cells (8)).

Device success was defined as successful placement of the TGT wire into the LAD with the administration of TGT therapy.

Hemodynamic Assessment: There was continuous measurement of hemodynamic parameters. Cardiac output was measured immediately before and after ischemia and pre-sacrifice. Hemodynamic stability and contractility were supported medically by IV dopamine, and epinephrine to maintain MAP above 50 mmg. Boluses of atropine (1 mg) and lidocaine (100 mg) were given as appropriate for treatment of arrhythmias. IV calcium and bicarbonate were given in appropriate doses during cardiopulmonary resuscitation. Each administration of these drugs was recorded as 1 arbitrary unit.

Trans Guide wire Therapy: TGT strategies included injection of 0.5 cc Evans blue or radiographic contrast to evaluate the distribution of injectate, nitroglycerine, heparinized saline or chloramphenicol. The latter was prepared as 10 mg in 0.5 cc of saline for TGT in addition to 40 mg/Kg IV. Injections were performed just prior to reperfusion. FIG. 10 (explained herein) shows an arrangement of syringes used for introduction of agent into the guide wire.

Echocardiography: Myocardial ejection fraction (EF) was quantitatively determined using left ventricular end-diastolic and end-systolic volumes. The volumes were calculated using the modified Simpsons' formula by 2 independent echocardiographers blinded to TGT therapy. Measurements were taken at baseline, end of ischemia and pre-sacrifice.

Western Blotting: After cardiac harvest the infarct border area was immediately frozen in liquid nitrogen. The remainder was stored in 10% formaldehyde for histology. Samples were homogenized in a buffer containing 50 mM Tris/HCl (pH 7.7), 100 mM sodium chloride, 1% Triton X-100, 10% Glycerol, 2.5 mM EDTA, 100 mM NaF and a protease inhibitor cocktail (Sigma). Homogenates were centrifuged for 10 min at 11500 g at 4° C. Supernatant protein concentration was measured using a BCA assay kit (Pierce Laboratories). Proteins (50 μg) were separated using SDS-polyacrylamide 7.5% gels for endothelial nitric oxide synthase (eNOS) and inducible nitric oxide synthase (iNOS) and 10% gels for Caspase 3. Molecular weight markers (Santa Cruz Biotechnology) and positive controls (BD Transduction laboratories) were treated in a similar manner. Electrophoresis lasted 1.15 h (Bio-Rad, Hercules, Calif.). Proteins were blotted by electro diffusion for 1.5 h at 80 mA on nitrocellulose membranes. They were blocked with Tris-buffered saline containing 5% (weight/volume) nonfat milk for 1 h and then blotted with primary iNOS, eNOS and Caspase-3 antibodies, 1/250 dilution, for 2 h at room temperatures. The membranes were extensively washed with Tris-buffered saline and 0.2% Tween-20 (TBST) and incubated for 1 h with goat anti rabbit antibodies conjugated with horseradish peroxidase (HPO), 1/2000 dilution, (Santa Cruz Biotechnology). After 3 TBST washings the immunocomplexes were developed using an enhanced HPO/luminol chemiluminescence reaction, and recorded photographically (Hyper film ECL; Amersham) by 10 s to 3 min exposure. Mouse macrophage +IFN/lysate for iNOS, human endothelial cells for eNOS and Jurkat cell lysate for Caspase-3 (BD Transduction laboratories) were used as positive controls, respectively. Quantification was performed using scanning densitometry with image J software.

TUNEL Staining: Terminal deoxy nucleotidyl transferase (TdT)-mediated dUTP nick end labelling was used for detection of apoptosis (Chemicon International, USA). Paraffin embedded tissue sections were fixed in 10% formalin then dewaxed in Xylene, taken through ethanol, incubated with Proteinase K, washed with distilled water, 3% hydrogen peroxide added. Finally, they were washed with PBS and incubated with TdT and stop/wash buffer. Digoxigenin dUTP was visualized by an antidigoxigenin peroxidase conjugate. A negative control using all reagents except TdT was performed in parallel. The brown apoptotic cell nuclei were detected by high power (×400) light microscopy. Ten optical fields consisting of approximately 500-1000 cells, were counted on each slide. The apoptosis index was defined as the percentage of apoptotic cells per 1000 cells.

Results:

Feasibility of Transguidewire Therapy: Two animals were studied with radiographic contrast TGT, 2 with Evans Blue TGT, 5 with saline TGT, and 5 with chloramphenicol TGT. All animals concurrently received TGT nitroglycerine. TGT device success in these animals was 100%. The TGT wire could be safely and easily positioned into the apical LAD. TGT wire luminal potency was maintained for the duration of the ischemic period. TGT injection into the downstream occluded LAD was successfully achieved instantaneously at the end of the ischemic period in all.

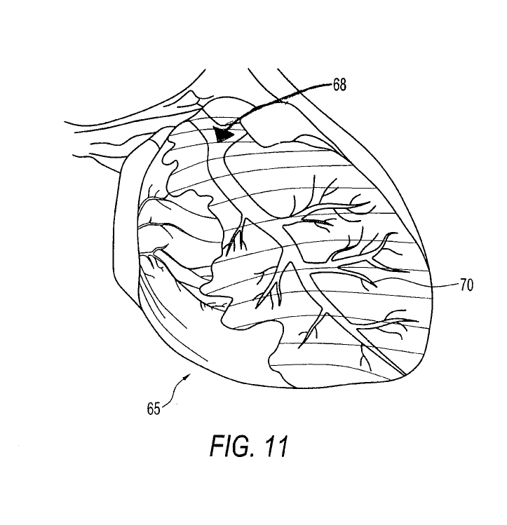

A 0.5 cc Evans Blue TGT injection graphically demonstrated the feasibility concept of Trans Guide wire Therapy (shown in sketch form in FIG. 11). In a heart 65, with ligature 68 with no staining proximal to the suture line Evans Blue perfused up to the lines of watershed with other coronaries demarcating the ischemic area at risk 70. A similar pattern of distribution was seen radiographically following TGT injection or radiopaque contrast medium.

Early in our experience 2 additional animals were studied with systemic IV chloramphenicol and nitroglycerine TGT injection. In the first of these animals the LAD surgical suture was initially non-occlusive. It was removed and a new suture reapplied. By the end of the experiment the distal wire tip had fractured. This led to a design advancement with extra crimping and soldering of the distal tip. No further structural problems were encountered in the other experiments. In the case of the second animal it was not possible to inject the TGT nitroglycerine at the end of the ischemic period because of wire luminal thrombosis. This led to protocol enhancement which stipulated that after successful placement the TGT wire lumen should be flushed with 500 IU of heparin (0.5 cc of 1000 IU/cc). No further thromboses were encountered. These 2 animals were not included in any further hemodynamic, echocardiographic or immunohistochemical analysis.

Survival and Hemodynamics: Of the chloramphenicol TGT treated animals all 5 survived the period of reperfusion to be sacrificed at 2 h. Of the Saline TGT-treated animals, 2 died during the reperfusion period. Both suffered terminal cardiac arrest despite extensive resuscitation attempts.

The saline and chloramphenicol TGT-treated animals had an equivalent rate pressure product (RPP) at baseline (Table 1).

TABLE 1

| | Baseline | End of ischemia | Pre-sacrifice |
|---|---|---|---|
| Chloram- | 3980.8 +/− 594.5 | 2969.5 +/− 687.2 | 3720.0 +/− 510.2* |
| phenicol | | | |
| Saline | 4808.4 +/− 731.5 | 3445 +/− 710.8 | 2685.7 +/− 2227.6** |
| p value | 0.11 | 0.4 | 0.02 |

*p = 0.21 when compared with baseline,
**p = 0.19 when compared with baseline.

With the onset of ischemia the overall mean RPP dropped from 4440.6,+/−768.4, to 3173.3+/−684.9 (p=0.002). The RPP at the end of ischemia was not significantly different between the two groups. However, pre-sacrifice, the chloramphenicol TGT group RPP had returned almost to baseline. This was in stark contrast to the saline TGT group where the RPP continued to deteriorate leading to an overall 44% drop (chloramphenicol vs. saline: 3720.0 (+/−510.2) vs. 2685.7 (+/−2227.6); p=0.02).

This difference in hemodynamic performance between the 2 groups was despite significantly more supportive therapy in saline TGT-treated animals (Table 2).

TABLE 2

| | Lidocaine | Dopamine | Epinephrine | Atropine | Bicarbonate | calcium | Total |
|---|---|---|---|---|---|---|---|
| Chloramphenicol | 4.5 | 1 | 1 | 2.5 | 1 | 0 | 10 |
| Saline | 9 | 2 | 16 | 6 | 3 | 6 | 42 |

Overall 42 arbitrary units of treatment were utilized in the saline animals relative to 10 arbitrary units in the chloramphenicol group (mean 7.0 (+/−5.1) vs. 1.67 (+/−1.6); p=0.028). All drug groups were required more often for support of the saline TGT animals.

Echocardiographic Assessment: At baseline both the saline and chloramphenicol TGT-treated animals had equivalent LV ejection fraction (43.9%+/−10.8 vs. 45.3%+/−12.0, p=0.82). After the 30 minute period of ischemia, chloramphenicol-treated animals had a larger reduction in EF but made the better recovery during reperfusion returning to near baseline (27.3%+/−3.8 to 43.3%+/−11.4, p<0.001). Saline-treated animals did not recover from ischemia with just minimal improvement of EF (35.1%+/−12.2 to 38.9%+/−7.0, p=0.52). Immunohistochemistry: The difference in Nos3/beta actin ratio for the chloramphenicol and saline groups may be non significant (chloramphenicol vs. saline 4.3±2.8 vs. 3.7±1.6, p=0.36). However, the concentration of caspase-3 may be significantly lower in the chloramphenicol group (chloramphenicol vs. saline 346±110 vs. 578±104, p=0.007). The TUNEL staining was weakly positive in multiple samples in both groups and therefore non-discriminatory between TGT with chloramphenicol or saline.

The foregoing description of specific embodiments is illustrative of the general nature of the subject matter claimed so that others can readily modify and/or adapt such embodiments for various applications without departing from the generic concepts presented herein. The claims hereof are to be understood to include without limitation all alternative embodiments and equivalents of the subject matter hereof. Phraseology and terminology employed herein are for the purpose of description and illustration and are not limiting. Where permissible by law, all references cited herein are incorporated by reference in their entirety. It will be appreciated that any aspects of the different embodiments disclosed herein may be combined with possible alternative embodiments, and alternative combinations of features and accordingly that the limitations of any one claim may be combined with the limitations of any other claim or claims without departing from the spirit and intention of this disclosure.

1. A hollow member for the intravascular delivery of an agent said member having an end and a delivery hole positioned relative to said end.

2. The member according to claim 1, wherein said member is filled with said agent.

3. The member according to claim 1 further comprising a marker.

4. The member according to claim 1, wherein said member is a guide wire.

5. An apparatus for performing an angioplasty, comprising a member according to claim 1.

6. The member according to claim 1, wherein said agent is selected from the group consisting of:
a) an inhibitor of superoxide production;
b) a platelet activation factor inhibitor;
c) a Caspase inhibitor; and
d) a promoter of NO production.

7. The member according to claim 1, wherein said intravascular delivery is in a cardiac blood vessel.

8. The member according to claim 1, wherein said intravascular delivery is in a blood vessel in the brain.

9. The member according to claim 1 for use in a human.

10. A catheter assembly comprising the member according to claim 1.

11. Apparatus for the intravascular delivery of an agent said apparatus comprising a fluid pressure source and a member, said member having an end and a delivery hole positioned relative to said end and said fluid pressure source being operatively connected to said member.

12. The apparatus according to claim 11, wherein said member is filled with said agent.

13. The apparatus according to claim 11, wherein said member has an associated marker.

14. The apparatus according to claim 11, wherein the apparatus is a catheter assembly.

15. The apparatus according to claim 11, wherein said member is a guide wire.

16. The apparatus according to claim 11, wherein said apparatus is adapted for performing an angioplasty.

17. The apparatus according to claim 11, wherein said agent is selected from the group consisting of:
a) an inhibitor of superoxide production;
b) a platelet activation factor inhibitor;
c) a Caspase inhibitor; and
d) a promoter of NO production.

18. The apparatus according to claim 11, wherein said intravascular delivery is in a cardiac blood vessel.

19. The apparatus according to claim 11, wherein said intravascular delivery is in a blood vessel in the brain.

20. The apparatus according to claim 11 for use in a human.

21. A method for reperfusing tissue, said method comprising locally delivering an agent to said tissue prior to reperfusing said tissue.

22. The method according to claim 21, wherein said method further comprises controlling the amount of said agent delivered to said tissue.

23. The method according to claim 21, further comprising exposing said tissue to said agent for a predetermined time period prior to said reperfusing.

24. The method according to claim 21, wherein said agent is delivered through an intravascular member.

25. The method according to claim 24, wherein said member is comprised in a catheter assembly.

26. The method according to claim 24, wherein said member includes a marker and a delivery hole and said method further comprises using said marker to determine the location of said delivery hole.

27. The method according to claim 24, wherein said method further comprises:
a) delivering said agent at a location relative to a vascular occlusion; and then
b) clearing said vascular occlusion.

28. The method according to claim 27, wherein said occlusion has first and second occlusion ends and said intravascular member is inserted through said occlusion from said first end and used to dispense said agent at said second end.

29. The method according to claim 21, wherein said member is a guide wire and said guide wire is comprised in an over the wire catheter assembly.

30. The method according to claim 29, wherein said guide wire has an end and, a delivery hole located relative to said end, and wherein said method further comprises positioning said member relative to said tissue.

31. The method according to claim 30, wherein said guide wire comprises a plurality of delivery holes.

32. The method according to claim 30, wherein said guide wire comprises a second marker and said method further comprises using said markers to position said member.

33. The method according to claim 21, wherein said agent is a chemical.

34. The method according to claim 21, wherein said agent is selected from the group consisting of:
a) an inhibitor of superoxide production;
b) a platelet activation factor inhibitor;
c) a Caspase inhibitor; and
d) a promoter of NO production.

35. The method according to claim 21, wherein the reperfusion is reperfusion of a cardiac blood vessel.

36. The method according to claim 21, wherein the reperfusion is in a blood vessel in the brain.

37. The method according to claim 21, wherein the tissue is human tissue.

38. A kit for reperfusing tissue, said kit comprising:
a) a hollow member suitable for intravascular delivery of an agent;
b) instructions to use said member to locally deliver an agent prior to reperfusing said tissue.

39. The kit according to claim 38, wherein said kit further comprises instructions to fill said member with said agent prior said reperfusing.

40. The kit according to claim 38, wherein said kit further comprises instructions to allow said agent to contact said tissue for a predetermined time prior to said reperfusion.

41. The use of a hollow member for the local delivery of an agent for
a) preventing; or
b) treating; or
c) preventing and treating a reperfusion injury.

42. The use according to claim 41, wherein said member is adapted to be insertable through a vascular lumen.

43. The use according to claim 41, wherein said member has a delivery hole and a marker positioned at a defined location relative to said delivery hole.

44. The use according to claim 41, wherein said use comprises filling said member with said agent.

45. The use according to claim 41, wherein said use comprises dispensing a known quantity of said agent through said member.

46. The use according to claim 41, wherein said use comprises allowing a determined time to pass between said delivery of said agent and reperfusing said tissue.

47. The use according to claim 41, wherein said member comprises a plurality of said markers and a plurality of said delivery holes.

48. The use according to claim 41, wherein said reperfusion is reperfusion of a cardiac blood vessel.

49. The use according to claim 41, wherein said reperfusion is reperfusion of a blood vessel in the brain.

50. The use according to claim 41, wherein said agent is a chemical.

51. The use according to claim 41, wherein said agent is selected from the group consisting of:
a) an inhibitor of superoxide production;
b) a platelet activation factor inhibitor;
c) a Caspase inhibitor; and
d) a promoter of NO production.

52. The use according to claim 41, wherein said hollow member further comprises a marker.

53. The use of a catheter assembly having a hollow guide wire for the reperfusion of tissue associated with the clearing of a vascular occlusion, wherein said using comprises locally delivering an agent through said guide wire prior to said clearing of said vascular occlusion.

54. The use according to claim 53, wherein said agent is selected from the group consisting of:
a) an inhibitor of superoxide production;
b) a platelet activation factor inhibitor;
c) a Caspase inhibitor; and
d) a promoter of NO production.

55. Use of a hollow member having an end, a delivery hole positioned proximate said end, and a marker positioned relative to said delivery hole to manufacture an apparatus for the local intravascular delivery of an agent in the removal of a vascular occlusion.

56. The use of a locally administered agent for
a) preventing; or
b) treating; or
c) preventing and treating reperfusion injury.

57. The use according to claim 56, wherein said agent is selected from the group consisting of:
a) an inhibitor of superoxide production;
b) a platelet activation factor inhibitor;
c) a Caspase inhibitor; and
d) a promoter of NO production.

58. The use according to claim 56, wherein said reperfusion occurs in a cardiac blood vessel.

59. The use according to claim 56, wherein said reperfusion occurs in a blood vessel in a brain.

60. The use according to claim 56, wherein said blood vessel is in a human.

61. The use of the member according to claim 1, to
a) prevent; or
b) treat; or
c) treat and prevent reperfusion injury.

62. The use according to claim 61, wherein said use occurs as part of an angioplasty procedure.

63. The use of a locally delivered agent for i) preventing; or ii) treating; or iii) preventing and treating reperfusion injury in a human cardiac blood vessel, said use comprising sequentially:
a) delivering a desired quantity of said agent at a desired intravascular location through a hollow intravascular member; and
b) waiting a desired time interval after said local delivery; and then
c) carrying out said reperfusion.

64. A method for clearing a vascular occlusion in a cardiac blood vessel, said method comprising sequentially:
a) exposing tissue affected by said occlusion to a desired quantity of an agent for a desired time; and then
b) reperfusing said tissue.

65. A method for clearing a vascular occlusion using a catheter assembly with a hollow guide wire, said method comprising sequentially:
a) passing said guide wire through said occlusion;
b) delivering a desired quantity of an agent through said guide wire;
c) waiting for a desired time; and
d) clearing said vascular occlusion.

66. A method for intravascularly delivering an agent for use in reperfusion, said method comprising:
a) filling a hollow member with said agent;
b) feeding said member through a desired blood vessel;
c) expelling a desired quantity of said agent from said member at a desired location.

67. A method for preparing tissues prior to reperfusion said method comprising delivering an agent to said tissue prior to reperfusing said tissue.

68. The method according to claim 67, wherein said method further comprises controlling the amount of said agent delivered to said tissue.

69. The method according to claim 67, further comprising exposing said tissue to said agent for a predetermined time period prior to said reperfusing.

70. The method according to claim 67, wherein said agent is delivered through an intravascular member.

71. The method according to claim 67, wherein said member is comprised in a catheter assembly.

72. The method according to claim 67, wherein said member includes a marker and a delivery hole and said method further comprises using said marker to determine the location of said delivery hole.

73. The method according to claim 67, wherein said method further comprises:
a) delivering said agent at a location relative to a vascular occlusion; and then
b) clearing said vascular occlusion.

74. The method according to claim 71, wherein said method comprises clearing a vascular occlusion and wherein said occlusion has first and second occlusion ends and said intravascular member is inserted through said occlusion from said first end and used to dispense said agent at said second end.

75. The method according to claim 71, wherein said member is a guide wire and said guide wire is comprised in an over the wire catheter assembly.

76. The method according to claim 75, wherein said guide wire has an end, and a delivery hole located relative to said end, and wherein said method further comprises positioning said member relative to said tissue to be reperfused.

77. The method according to claim 76, wherein said member further comprises a marker and said method further comprises using said marker to position said member.

78. The method according to claim 76, wherein said guide wire comprises a plurality of delivery holes.

79. The method according to claim 76, wherein said guide wire comprises a second marker.

80. The method according to claim 67, wherein said agent is a chemical.

81. The method according to claim 67, wherein said agent is selected from the group consisting of:
a) an inhibitor of superoxide production;
b) a platelet activation factor inhibitor;
c) a Caspase inhibitor; and
d) a promoter of NO production.

82. The method according to claim 67, wherein the reperfusion is reperfusion of a cardiac blood vessel.

83. The method according to claim 67, wherein the reperfusion is in a blood vessel in the brain.

84. The method according to claim 67, wherein the tissue is human tissue.
